(19) United States
(12) Patent Application Publication
(10) Pub. No.: US 2015/0299649 A1
(43) Pub. Date: Oct. 22, 2015

(54) HIGHLY EFFICIENT PRODUCTION OF LACTIC ACID FROM HEMICELLULOSE SUGARS BY NEWLY ISOLATED THERMOPHILIC BACILLUS COAGULANS STRAINS

(71) Applicant: AGENCY FOR SCIENCE, TECHNOLOGY AND RESEARCH, Singapore (SG)

(72) Inventors: Jinchuan Wu, Jurong Island (SG); Linda Ye, Jurong Island (SG); Xingding Zhou, Jurong Island (SG)

(21) Appl. No.: 14/647,053

(22) PCT Filed: Nov. 22, 2013
(86) PCT No.: PCT/SG2013/000497

§ 371 Date: May 22, 2015

(30) Foreign Application Priority Data
Nov. 23, 2012 (SG) 201208653-4

(51) Int. Cl. C12N 1/20; C12R 1/07; C12P 7/56

(52) U.S. Cl. CPC C12N 1/20; C12P 7/56; C12R 1/07

(57) ABSTRACT

The present invention relates to newly isolated Bacillus coagulans strains C106, JI 12 and WCPlO-4, cost effective methods for producing lactic acid using the strains as well as seed inoculums and kits comprising the strains. Optically pure L-lactic acid was produced from xylose or glucose at high product titers (>200 g/L) and productivities (4.2 g/L/h on xylose and 4.6 g/L/h on glucose) at 50° C. When an acid hydrolysate (85 g sugars/L) of empty fruit bunch of oil palm trees was used, the L-lactic acid titer, yield and productivity reached 84.5 g/L, 97% and 1.84 g/L/h, respectively. A one-step, one-pot open fermentation was also developed to produce optically pure L-lactic acid from starch. The final lactic acid titer and productivity were as high as 202.0 g/L and 5.4 g/L/h, respectively. No glucose remained in the broth and the lactic acid yield reached 98%.

### TECHNICAL FIELD

The present invention generally relates to newly isolated thermophilic Bacillus coagulans strains, methods for producing lactic acid using the strains as well as seed inoculums and kits comprising the strains.

### BACKGROUND

### Lactic Acid from Plant Originated Biomass

Lactic acid has wide applications in food, feed, cosmetics and textile industries as well as in producing poly lactic acid (PLA), a promising biodegradable polymer. Lactic acid is commercially produced from glucose, starch or sucrose. These raw materials contribute to a significant proportion of lactic acid production cost. To reduce the cost of feedstock and avoid competition with foods, the use of cheap, abundant and renewable lignocellulose as an alternative feedstock has received much attention. Lignocellulose is composed of cellulose (30-50%), hemicellulose (20-40%) and lignin (10-30%). In theory, all the sugar components of lignocellulose can be utilized as carbon sources for microbial fermentation, but xylose, the major component of hemicellulose, cannot be efficiently metabolized by most lactic acid bacteria. For the microbes that ferment pentoses, the phosphoketolase pathway is usually adopted, yielding equal molar amount of acetic acid and lactic acid (Patel, M. A., Ou, M. S., Harbrucker, R., Aldrich, H. C., et al., Isolation and characterization of acid-tolerant, thermophilic bacteria for effective fermentation of biomass-derived sugars to lactic acid. Applied and Environmental Microbiology 2006, 72, 3226-3235). Therefore, fermentation of pentose sugars from hemicelloluse remains a major obstacle for economical production of lactic acid from plant-originated biomass.

Lactic acid is commercially produced from starchy materials using mesophilic lactic acid bacteria in rich medium, which needs several steps including gelatinization and liquefaction at 90-130° C., enzymatic saccharification at 50-70° C., sterilization at 121° C. and fermentation at 30-40° C. Recently, direct starch fermentation using amylolytic strains and by simultaneous saccharification and fermentation (SSF) have received attention for lactic acid production, but the lactic acid titer of amylolytic bacteria is usually low due to the low expression level of amylase. SSF can usually give a high lactic acid titer but medium sterilization is still essential for mesophilic lactic acid bacteria, which not only consumes energy but also causes a sugar loss.

Bacillus coagulans for Fermenting Sugars

In recent years, Bacillus species, especially Bacillus coagulans, have attracted much attention owing to their strong ability to ferment pentose sugars, specifically xylose, to optically pure L-lactic acid. These bacterial strains convert pentoses to lactic acid homo-fermentatively via the pentose phosphate pathway (Patel et al.). Bacillus coagulans strains are moderately thermophilic and capable of growing at temperatures above 45° C. Fermentation at such high temperatures reduces the risk of microbial contamination. Using these strains, lactic acid can be produced under unsterile conditions, which decreases the expenses for lactic acid fermentation in industrial scale. Besides, B. coagulans strains can grow in a simple medium with lower requirement of nutrients compared to Lactobacilli (Patel et al.), the bacterial species that are widely used for industrial lactic acid production. However, the lower titer and productivity of the known B. coagulans strains limit their industrial applications for lactic acid production (WO2004063382, WO2011053576, EP1409642, WO2005086670).

Lactic acid bacteria (LAB) are extensively utilized for lactic acid production. However, most LAB are mesophilic (30-41° C.), so medium sterilization is essential to avoid microbial contamination, which not only causes a sugar loss but also increases the production cost. If lactic acid fermentation is able to be performed at higher temperatures (≧50° C.), the medium sterilization would become unnecessary as the risk of microbial contamination would be minimized. In addition, producing lactic acid at higher temperatures favors simultaneous saccharification and fermentation (SSF) of starch or cellulose as amylases and cellulases are usually more active at higher temperatures.

Furthermore several Bacillus coagulans strains exhibited strong ability of producing L-lactic acid from both hexose and pentose sugars with higher lactic acid titers (>150 g/L) but lower productivities (<3 g/L/h) (Ou M S, Ingram L O, Shanmugam K T (2011) L: (+)-Lactic acid production from non-food carbohydrates by thermotolerant Bacillus coagulans. J Ind Microbiol Biotechnol 38(5): 599-605; 2011; Qin J, Zhao B, Wang X, Wang L, Yu B, Ma Y, Ma C, Tang H, Sun J, Xu P (2009) Non-sterilized fermentative production of polymer-grade L-lactic acid by a newly isolated thermophilic strain Bacillus sp. 2-6. PLoS One 4(2): e4359).

In past several years, a number of thermotolerant or thermophilic (50-60° C.) lactic acid producers such as Bacillus coagulans 36D1 and P4-102B (Patel M A, Ou M S, Harbrucker R, Aldrich H C, Buszko M L, Ingram L O, Shanmugam K T (2006) Isolation and characterization of acid-tolerant, thermophilic bacteria for effective fermentation of biomass-derived sugars to lactic acid. Appl Environ Microbiol 72(5): 3228-35), Bacillus sp. 2-6 (Qin J, Zhao B, Wang X, Wang L, Yu B, Ma Y, Ma C, Tang H, Sun J, Xu P (2009) Non-sterilized fermentative production of polymer-grade L-lactic acid by a newly isolated thermophilic strain Bacillus sp. 2-6. PLoS One 4(2): e435), Bacillus sp. XZL9 (Wang L, Zhao B, Liu B, Yu B, Ma C, Su F, Hua D, Li Q, Ma Y, Xu P (2010) Efficient production of L-lactic acid from corncob molasses, a waste by-product in xylitol production, by a newly isolated xylose utilizing Bacillus sp. strain. Bioresour Technol), Bacillus licheniformis TY7 (Sakai K, Yamanami T (2006) Thermotolerant Bacillus licheniformis TY7 produces optically active L-lactic acid from kitchen refuse under open condition. J Biosci Bioeng 102(2): 132-4), Bacillus coagulans SIM-7 (Michelsona T, Kaskb K, Jogia E, Talpsepa E, Suitsoa I, Nurka A (2006) 1(+)-Lactic acid producer Bacillus coagulans SIM-7 DSM 14043 and its comparison with Lactobacillus delbrueckii ssp. lactis DSM 20073. Enzyme Microb Technol 39(4): 861-867), Bacillus coagulans MXL-9 (Walton S L, Bischoff K M, van Heiningen A R, van Walsum G P (2010) Production of lactic acid from hemicellulose extracts by Bacillus coagulans MXL-9. J Ind Microbiol Biotechnol 37(8): 823-30), Bacillus coagulans CCM 4318 (Rosenberg M, Rebros M, Kristofikova L, Malatova K (2005) High temperature lactic acid production by Bacillus coagulans immobilized in LentiKats. Biotechnol Lett 27(23-24): 1943-7) and Bacillus licheniformis BL1 (Wang Q, Zhao X, Chamu J, Shanmugam K T (2011) Isolation, characterization and evolution of a new thermophilic Bacillus licheniformis for lactic acid production in mineral salts medium. Bioresour Technol 102(17): 8152-8) have been isolated from nature. Most of them belong to Bacillus species and produce only L-lactic acid. There is one exception that a thermotolerant D-lactic acid producer Lactobacillus delbrueckii subsp. lactis QU 41 was recently reported (Tashiro Y, Kaneko W, Sun Y, Shibata K, Inokuma K, Zendo T, Sonomoto K (2011) Continuous D-lactic acid production by a novel thermotolerant Lactobacillus delbrueckii subsp. lactis QU 41. Appl Microbiol Biotechnol 89(6): 1741-50). The performance of all these strains is not satisfying in all regards.

Therefore there is still a need for cost-effective processes for producing lactic acid from lignocellulose sugars using newly isolated Bacillus coagulans strains with very high lactic acid titer, productivity and yield. Further, to promote commercial applications, more powerful thermophilic strains need to be developed to produce lactic acid with both high titer and high productivity. The lower glucose tolerance of reported thermophilic strains might be one of the reasons leading to the lower productivity.

Production of lactic acid using thermophilic Bacillus coagulans strains from lignocelluloses sugars has received much attention in recent years owing to many advantages of the thermophilic strains compared to the conventional mesophilic lactic acid bacteria. However, the lactic acid titers and productivities of the reported Bacillus coagulans strains are not high enough to meet the requirement for industrial production. Therefore, isolating new thermophilic B. coagulans strains and developing new processes to improve the lactic acid titer and productivity are essential for industrial production of lactic acid from lignocelulose sugars.

### SUMMARY OF THE INVENTION

According to the invention there have been isolated new strains of Bacillus coagulans which can directly or in form of their functional mutants overcome the deficiencies of the cited references above.

### DEFINITIONS

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

Unless the context requires otherwise or specifically stated to the contrary, integers, steps, or elements of the invention recited herein as singular integers, steps or elements clearly encompass both singular and plural forms of the recited integers, steps or elements.

Throughout this specification and claims, unless the context requires otherwise, the word “comprise”, or variations such as “comprises” or “comprising”, will be understood to imply the inclusion of a stated step or element or integer or group of steps or elements or integers, but not to the exclusion of any other step or element or integer or group of elements or integers. Thus, in the context of this specification, the term “comprising” means “including peripherally, but not necessarily solely”.

When describing the compounds, compositions, methods and processes of the invention, the following terms have the following meanings unless otherwise indicated. Additionally, as used herein, the singular forms “a,” “an” and “the” include the corresponding plural forms unless the context of use clearly dictates otherwise.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

### DISCLOSURE OF INVENTION

Detailed aspects of the invention will now be disclosed.

According to a first aspect, there are newly isolated strains of Bacillus coagulans and their functional mutants. The object of the invention are Bacillus coagulans strain C106 deposited under Accession No. PTA-13254 or a functional mutant thereof, Bacillus coagulans strain JI 12 deposited under Accession No. PTA-13253 or a functional mutant thereo and Bacillus coagulans strain WCP10-4 deposited under Accession No. PTA-13255 or a functional mutant thereof. Mutants are strains arising or resulting from mutation. A functional mutant shows essentially the same function as the strain it is derived from by mutation. Object of the invention are also the biological pure strains. A biological pure strain is group of identical individuals that always produce offspring of the same phenotype when intercrossed.

Surprisingly the newly isolated strains show several improvements over previously known strains as shown in the examples. They show very high titer, productivity and yield in producing L-lactic acid from xylose and hemicellulose sugars. No medium sterilization is required. And reduced cost of nitrogen sources in the fermentation medium is given.

For glucose fermentation Bacillus coagulans strain JI 12 converted all sugars homefermenttatively with a high productivity. Bacillus coagulans WCP10-4 converts corn starch to optically pure L-lactic acid with high titer, yield and productivity in a one-pot, one-step process without the requirement of sterilizing the medium and fermenter prior to fermentation (so-called open fermentation).

Although Bacillus coagulans WCP10-4 was isolated using glucose as the carbon source, it has been proven to be able to convert pentose sugars such as xylose and arabinose to L-lactic acid homofermentatively, but with a slightly lower productivity on pentose than on glucose.

Advantageously, the newly isolated strains can be used for producing lactic acid with very high lactic acid titers, productivity and yield.

In a second aspect, there is provided a method of producing lactic acid from a carbohydrate source, comprising the steps of:
(i) contacting said carbohydrate source with at least one Bacillus coagulans strain selected from the group consisting of Bacillus coagulans strain C106 deposited under Accession No. PTA-13254; Bacillus coagulans strain JI 12 deposited under Accession No. PTA-13253; Bacillus coagulans strain WCP10-4 deposited under Accession No. PTA-13255; and a functional mutant thereof to form lactic acid; and
(ii) isolating said lactic acid.

Advantageously, the method is used where the carbohydrate source is selected from the group consisting of lignocellulose, hemicellulose, D/L-glucose, D/L-galactose, D/L-mannose, D/L-arabinose, D/L-lyxose, D/L-ribose, D/L-xylose, D/L-ribulose, D/L-xylulose, saccharides and polysaccharides thereof, for example starch. Most advantageously the carbohydrate source is L-xylose.

Advantageously, in the method according to the third aspect, at least one Bacillus coagulans strain C106 deposited under Accession No. PTA-13254 is used.

Advantageously, it is also possible to incubate two Bacillus coagulans strains with the carbohydrate source in the method according to the third aspect. Most advantageously these two strains are C106 deposited under Accession No. PTA-13254 and JI 12 deposited under Accession No. PTA-13253.

Advantageously, the method of the third aspect according to the invention is used to produce L-lactic acid. Most advantageously the isolated L-lactic acid has an optical purity greater than 99% and/or the titer of this L-lactic acid is in the range selected from the group consisting of 50 to 150 g/L, 60 to 140 g/L, 70 to 130 g/L, 80 to 130 g/L, 90 to 130 g/L, and 100 to 130 g/L.

Advantageously, production of L-lactic acid according to the third aspect of the invention is in the range selected from the group consisting of 1 to 8 g/L/hour, 2 to 7 g/L/hour, 3 to 6 g/L/hour, and 4 to 5 g/L/hour and/or the yield of isolated L-lactic acid is greater than 90%.

Advantageously, Bacillus coagulans strain is incubated with said carbohydrate source at a temperature of from 50-55° C. Thermophilic strains are an object of this invention.

Advantageously, the incubation time of the Bacillus coagulans strain with the carbohydrate source is for a period of time from 24 to 72 hours.

In a fourth aspect, there are provided the individual seed inoculums or their functional mutants of all strains according to the first aspect of the invention. The use of the strain to inoculate a new culture is therefore another object of the invention.

Advantageously, the seed inoculums according to the forth aspect further comprise a carrier. Most advantageously, the carrier is water.

In a fifth aspect, there is provided a kit of at least one of the strains according to the first aspect of the invention together with instructions for use. Advantageously, the kit comprises at least one inoculum according the forth aspect of the invention together with instructions. A kit is defined as a set of articles or implements used for a specific purpose. Preferably, the purpose of the kits according to the invention is the intended use for producing lactic acid.

In a sixth aspect, there is provided a one-pot and/or one-step process for producing L-lactic acid under non-sterilized conditions using at least one Bacillus coagulans strain selected from the group consisting of Bacillus coagulans strain C106 deposited under Accession No. PTA-13254; Bacillus coagulans strain JI 12 deposited under Accession No. PTA-13253; Bacillus coagulans strain WCP10-4 deposited under Accession No. PTA-13255; and a functional mutant thereof.

Advantageously, in the process according to the sixth aspect at least one Bacillus coagulans strain is incubated with a carbohydrate source at a temperature ranging from 50-70° C. and/or the source is saccharides or polysaccharides such as starch.

### BRIEF DESCRIPTION OF DRAWINGS

The accompanying drawings illustrate a disclosed embodiment and serve to explain the principles of the disclosed invention. It is to be understood, however, that the drawings are designed for purposes of illustration only, and not as a definition of the limits of the invention.

FIG. 1 Batch fermentation of xylose by B. coagulans JI12

FIG. 2 Batch fermentation of xylose by B. coagulans C106

FIG. 3. Batch fermentation of xylose by B. coagulans WCP10-4

FIG. 4 Fed batch fermentation of xylose by Bacillus coagulans JI12

FIG. 5 Fed batch fermentation of xylose by Bacillus coagulans C106

FIG. 6 Fermentation of glucose by B. coagulans JI12

FIG. 7 Fermentation of glucose by B. coagulans C106

FIG. 8 Fermentation of glucose by B. coagulans WCP10-4

FIG. 9 Fermentation of EFB acid hydrolysate by B. coagulans strain JI12

FIG. 10 Simultaneous saccharification and fermentation of corn starch by B. coagulans strain WCP10-4

FIG. 11. Profiles of L-lactic acid production from glucose by Bacillus coagulans WCP10-4 in a 2 L fermenter containing 1 L no-sterilized medium (glucose 240 g/L, Yeast extract 20 g/L) at pH6.0, 200 rpm, 50° C.

FIG. 12 Profiles of simultaneous saccharification and fermentation to L-lactic acid from corn starch by Bacillus coagulans WCP10-4 in a 2 L fermenter containing 1 L no-sterilized medium (corn starch 200 g/L, yeast extract 20 g/L, α-amylase 0.5 mL, glucoamylase 5 mL) at pH5.5, 200 rpm, 50° C.

FIG. 13 Profiles of simultaneous saccharification and fermentation to L-lactic acid from corn starch by Bacillus coagulans WCP10-4 in a 2 L fermenter containing 1 L no-sterilized medium(corn starch 200 g/L, yeast extract 20 g/L, α-amylase 0.5 mL, glucoamylase 5 mL) at pH6.0, 200 rpm, 50° C.

### EXAMPLES

Non-limiting examples of the invention will be further described in greater detail, which should not be construed as in any way limiting the scope of the invention.

### First Set of Examples

Materials and Chemicals

Hemicellulose hydrolysate was prepared in our lab by acid-catalyzed hydrolysis of empty fruit bunch (EFB) of oil palm trees. EFB was kindly provided by Wilmar International Limited, Singapore. Other chemicals were of an analytical grade and obtained commercially.

Corn starch (starch content 100%) was purchased from Sigma-Aldrich. α-Amylase (Liquozyme SC DC, 240 KNU/g, 1 KNU is defined as the amount of enzyme that breaks down 5.26 g of starch per hour under Novozyme's assay conditions) and glucoamylase (Spirizyme Fuel, 750 AGU/g, 1 AGU is defined as the amount of enzyme that hydrolyzes 1 μmol of maltose per minute under specified conditions) were gifts from Novozymes, Denmark.

Culture Media

The liquid minimal medium for enrichment of xylose-fermenting bacteria from soil samples contained (per liter) 10 g of xylose and 10 g of yeast extract at pH 6.0. The solid minimal medium plates for screening xylose-fermenting microbes had the same compositions with the liquid medium but supplemented with 15 g of agar and 0.02% of Bromocresol Green which was used as a pH indicator for acid production. The liquid mineral salts medium (MSM) for cultivating the isolates contained (per liter): 2 g of (NH₄)₂SO₄, 2 g of KH₂PO₄, 2 g of NaCl, 0.2 g of MgSO4.7H₂O, 0.05 g of MnSO₄.7H₂O, 0.01 g of FeSO₄.7H₂O, 10 g of yeast extract, 50 g of xylose and 3% of CaCO₃, which was used as a neutralizing agent for lactic acid. The pH was adjusted to 6.0.

Procedures for Screening Thermophilic Lactic Acid Bacteria

Soil samples (2 g) were enriched in 25 ml of liquid minimal medium at 50° C. for overnight. The suspensions were then serially diluted and spread onto solid minimal medium plates. The plates were then kept in an incubator at 50° C. for 1-2 days until the appearance of colonies with clear colour change. The colonies were then picked up and cultivated in 2 ml of liquid mineral salts medium at 50° C. for 2 days. Samples (1 ml) were taken for HPLC analysis to quantify the consumption of xylose and production of lactic acid. The colonies with high lactic acid production were purified by streaking on new solid minimal medium plates repeatedly. The productivity lactic acid was defined as the amount of lactic acid produced per liter per hour.

Identification of Isolated Strains

The 16S rDNA of the selected isolates was amplified by PCR using the primer pairs, F27: 5′-AGAGTTTGATCCTGGCTCAG-3′ and 1492R: 5′-GGTTACCTTGTTACGACTT-3′, and the, L-lactate dehydrogenase gene of isolate JI12 was amplified by PCR using the primer pairs, B. coagulans ldhLF: 5′-ATGAAAAAGGTCAATCGTATTGCAGTG-3′ and B. coagulans ldhLR: 5′-TTACAATACAGGTGCCATCGTTT-3′. All amplified DNA fragments were sequenced and analyzed with the NCBI nucleotide database to identify the strains.

Results

Screening and Identification of Thermophilic Lactic Acid Bacteria

Three isolates (JI12, C106 and WCP10-4) with the highest lactic acid productivities on xylose were purified and identified.

The 16S rDNA sequence of isolate JI12 was as follows:

AAGGTTACCTCACCGACTTCGGGTGTTACAAACTCTCGTGGTGTGACGGG
CGGTGTGTACAAGGCCCGGGAACGTATTCACCGCGGCATGCTGATCCGCG
ATTACTAGCGATTCCGGCTTCATGCAGGCGGGTTGCAGCCTGCAATCCGA
ACTGGGAATGGTTTTCTGGGATTGGCTTAACCTCGCGGTCTCGCAGCCCT
TTGTACCATCCATTGTAGCACGTGTGTAGCCCAGGTCATAAGGGGCATGA
TGATTTGACGTCATCCCCACCTTCCTCCGGTTTGTCACCGGCAGTCACCT
TAGAGTGCCCAACTGAATGCTGGCAACTAAGGTCAAGGGTTGCGCTCGTT
GCGGGACTTAACCCAACATCTCACGACACGAGCAAACAAAAACCATGCAC
CACCTGTCACTCTGTCCCCCGAAGGGGAAGGCCCTGTCTCCAGGGGGTCA
GAGGATTCAAGACCTGGAAAGGTTCTTCGCGTTCTTAGAATAAAACCACA
TGCTCCACCGCTTGTGCGGGCCCCCGTAAATTCCTTTGAGTTTCAGCCTT
GCGGCCGTACTCCCCAGGCGGAGTGCTTAATGCGTTAGCTGCAGCACTAA
AGGGCGGAAACCCTCTAACACTTAGCACTCATCGTTTACGGCGGGGACTA
CCAGGGTATCTAATCCTGTTTGCTCCCCACGCTTTCGCGCCTCAGCGTCA
GTTACAGACCAGAGAGCCGCCTTCGCCACTGGTGTTCCTCCACATCTATA
CGCATTTCACCGATACACGTGGAATTCCACTCTCCTCTTCTGCACTCAAG
CCTCCCAGTTTCCAATGACCGCTTGCGGTTGAGCCGCAAGATTTCACATC
AGACTTAAGAAGCCGCCTGCGCGCGCTTTACGCCCAATAATTCCGGACAA
CGCTTGCCACATACGTATTACCGCGGCTGCTGGCACGTAGTTAGCCGTGG
CTTTCTGGCCGGGTACCGTCAAGGCGCCGCCCTGTTCGAACGGCA

The L-lactate dehydrogenase gene sequence of isolate JI12 was as follows:

ATGAAAAAGGTCAATCGTATTGCAGTGGTTGGAACGGGTGCAGTTGGTAC
AAGTTACTGCTACGCCATGATTAATCAGGGTGTTGCAGAAGAGCTTGTTT
TAATCGATATTAACGAAGCAAAAGCAGAAGGGGAAGCCATGGACCTGAAC
CACGGCCTGCCATTTGCGCCTACGCCGACCCGCGTTTGGAAAGGCGATTA
TTCCGATTGCGGCACTGCCGATCTTGTTGTCATTACGGCAGGTTCCCCGC
AAAAACCGGGCGAAACAAGGCTTGATCTTGTTTCCAAAAACGCAAAAATT
TTTAAAGGCATGATTAAGAGCATCATGGACAGCGGCTTTAACGGGATTTT
TCTTGTTGCCAGCAACCCGGTTGACATTTTGACATATGTAACTTGGAAAG
AGTCCGGCCTGCCGAAAGAACATGTTATCGGTTCGGGCACAGTGCTTGAC
TCCGCGCGTCTCCGCAACTCTTTGAGCGCCCAATTTGGAATTGACCCGCG
CAATGTGCATGCTGCGATTATCGGCGAACACGGCGATACGGAACTTCCGG
TATGGAGCCATACAAATATCGGTTACGATACGATTGAAAGCTATCTACAA
AAAGGAATTATTGACGAAAAGACGTTAGATGACATTTTTGTCAATACGAG
AGATGCGGCTTATCATATTATTGAACGAAAAGGGGCCACATTTTACGGCA
TCGGGATGTCCCTGACCCGGATTACAAGGGCAATCCTGAACAATGAAAAC
AGCGTATTGACGGTCTCTGCATTTCTTGAAGGCCAATACGGAAACAGCGA
TGTGTACGTTGGCGTTCCGGCCATCATCAATCGCCAGGGCATCCGTGAAG
TGGTTGAAATCAAACTGAACGAAAAAGAACAGGAACAGTTCAATCATTCT
GTAAAAGTGCTAAAAGAAACGATGGCACCTGTATTGTAA

After blasting in NCBI, it was found that the 16S rDNA of isolate JI12 had 98.5% identities with that of Bacillus coagulans strains SP9, 36D1, 2-6, SKU12, NRIC1526 and T5. The L-lactate dehydrogenase gene of isolate JI12 also showed high identities (94.1%-100%) with that of other Bacillus coagulans strains. Therefore, the isolate JI 12 was identified and named as Bacillus coagulans JI12.

The 16S rDNA sequence of isolate C106 was as follows:

TACCTCACCGACTTCGGGTGTTACAAACTCTCGTGGTGTGACGGGCGGTG
TGTACAAGGCCCGGGAACGTATTCACCGCGGCATGCTGATCCGCGATTAC
TAGCGATTCCGGCTTCATGCAGGCGGGTTGCAGCCTGCAATCCGAACTGG
GAATGGTTTTCTGGGATTGGCTTAACCTCGCGGTCTCGCAGCCCTTTGTA
CCATCCATTGTAGCACGTGTGTAGCCCAGGTCATAAGGGGCATGATGATT
TGACGTCATCCCCACCTTCCTCCGGTTTGTCACCGGCAGTCACCTTAGAG
TGCCCAACTGAATGCTGGCAACTAAGGTCAAGGGTTGCGCTCGTTGCGGG
ACTTAACCCAACATCTCACGACACGAGCTGACGACAACCATGCACCACCT
GTCACTCTGTCCCCCGAAGGGGAAGGCCCTGTCTCCAGGGAGGTCAGAGG
ATGTCAAGACCTGGTAAGGTTCTTCGCGTTGCTTCGAATTAAACCACATG
CTCCACCGCTTGTGCGGGCCCCCGTCAATTCCTTTGAGTTTCAGCCTTGC
GGCCGTACTCCCCAGGCGGAGTGCTTAATGCGTTAGCTGCAGCACTAAAG
GGCGGAAACCCTCTAACACTTAGCACTCATCGTTTACGGCGTGGACTACC
AGGGTATCTAATCCTGTTTGCTCCCCACGCTTTCGCGCCTCAGCGTCAGT
TACAGACCAGAGAGCCGCCTTCGCCACTGGTGTTCCTCCACATCTCTACG
CATTTCACCGCTACACGTGGAATTCCACTCTCCTCTTCTGCACTCAAGCC
TCCCAGTTTCCAATGACCGCTTGCGGTTGAGCCGCAAGATTTCACATCAG
ACTTAAGAAGCCGCCTGCGCGCGCTTTACGCCCAATAATTCCGGACAACG
CTTGCCACCTACGTATTACCGCGGCTGCTGGCACGTAGTTAGCCGTGGCT
TTCTGGCCGGGTACCGTCAAGGCGCCGCCCTGTTCGAACGGCACTTGTTC
TTCCCCGGCAACAGAGTTTTACGACCCGAAGGCCTTCTTCACTCACGCGG
CGTTGCTCCGTCAGACTTTCGTCCATTGCGGAAGATTCCCTACTGCTGCC
TCCCGTAGGAGTTTGGGCCGTGTCTCAGTCCCAATGTGGCCGATCACCCT
CTCAGGTCGGCTACGCATCGTTGCCTTGGTGAGCCGTTACCCCACCAACT
AGCTAATGCGCCGCGGGCCCATCTGTAAGTGACAGCAAAAGCCGTCTTTC
CTTTTTCCTCCATGCGGAGGAAAAAACTATCCGGTATTAGCCCCGGTTTC
CCGGCGTTATCCCGATCTTACAGGCAGGTTGCCCACGTGTTACTCACCCG
TCCGCCGCTAACCTTTTAAAAGCAAGCTTTTAAAAGG

The blasting in NCBI showed that the 16S rDNA of isolate C106 was highly homologous (99.9%) to that of Bacillus coagulans strains SP9, NRIC1526, T5, 36D1 and 001RC. Therefore, this isolate was identified and named as Bacillus coagulans C106.

The 16S rDNA sequence of isolate WCP10-4 was as follows:

GACCTTTTAAAAGCTTGCTTTTAAAAGGTTAGCGGCGGACGGGTGAGTAA
CACGTGGGTAACCTGCCTGTAAGATCGGGATAACGCCGGGAAACCGGGGC
TAATATCGGATAGTTTTTTCCTCCGCATGGAGGAAPAAGGAAAGACGGCT
TTTGCTGTCACTTACAGATGGGCCCGCGGCGCATTAGCTAGTTGGTGGGG
TAACGGCTCACCAAGGCAACGATGCGTAGCCGACCTGAGAGGGTGATCGG
CCACATTGGGACTGAGACACGGCCCAAACTCCTACGGGAGGCAGCAGTAG
GGAATCTTCCGCAATGGACGAAAGTCTGACGGAGCAACGCCGCGTGAGTG
AAGAAGGCCTTCGGGTCGTAAAACTCTGTTGCCGGGGAAGAACAAGTGCC
GTTCGAACAGGGCGGCGCCTTGACGGTACCCGGCCAGAAAGCCACGGCTA
ACTACGTGCCAGCAGCCGCGGTAATACGTAGGTGGCAAGCGTTGTCCGGA
ATTATTGGGCGTAAAGCGCGCGCAGGCGGTTTCTTAAGTCTGATGTGAAA
TCTTGCGGCTCAACCGCAAGCGGTCATTGGAAACTGGGGGGCTTGAGTGC
AGAAGAGGAGAGTGGAATTCCACGTGTAGCGGTGAAATGCGTAGAGATGT
GGAGGAACACCAGTGGCGAAGGCGGCTCTCTGGTCTGTAACTGACGCTGA
GGCGCGAAAGCGTGGGGAGCAAACAGGATTAGATACCCTGGTAGTCCACG
CCGTAAACGATGAGTGCTAAGTGTTAGAGGGTTTCCGCCCTTTAGTGCTG
CAGCTAACGCATTAAGCACTCCGCCTGGGGAGTACGGCCGCAAGGCTGAA
ACTCAAAGGAATTGACGGGGGCCCGCACAAGCGGTGGAGCATGTGGTTTA
ATTCGAAGCAACGCGAAGAACCTTACCAGGTCTTGACATCCTCTGACCTC
CCTGGAGACAGGGCCTTCCCCTTCGGGGGACAGAGTGACAGGTGGTGCAT
GGTTGTCGTCAGCTCGTGTCGTGAGATGTTGGGTTAAGTCCCGCAACGAG
CGCAACCCTTGACCTTAGTTGCCAGCATTCAGTTGGGCACTCTAAGGTGA
CTGCCGGTGACAAACCGGAGGAAGGTGGGGATGACGTCAAATCATCATGC
CCCTTATGACCTGGGCTACACACGTGCTACAATGGATGGTACAAAGGGCT
GCGAGACCGCGAGGTTAAGCCAATCCCAGAAAACCATTCCCAGTTCGGAT
TGCAGGCTGCAACCCGCCTGCATGAAGCCGGAATCGCTAGTAATCGCGGA
TCAGCATGCCGCGGTGAATACGTTCCCGGGCCTTGTACACACCGCCCGTC
ACACCACGAGAGTTTGTAACACCCGAAGTCGGTGAGGTAACCTTTACGGA
GCCAGCCGCCGAAGGTGGGACAGATGATTGGGGTGAAGTC

The blasting in NCBI showed that the 16S rDNA of isolate WCP10-4 had an identity of 100%, 99.9%, 99.8%, 99.8%, 99.7% and 99.7% with that of Bacillus coagulans strains 36D1, T3, NBRC 12583, 2-6, SL5 and ATCC 7050, respectively. Therefore, this isolate was named Bacillus coagulans WCP10-4.

Lactic Acid Production on Xylose by B. coagulans J112, C106 and WCP10-4

Growing in mineral salts medium supplemented with 1-2% of yeast extract and different concentrations of xylose, all three B. coagulans strains, JI12, C106 and WCP10-4, were shown to be able to produce lactic acid homo-fermentatively with negligible levels of acetic acid detected (<1 g/L). The optical purity of lactic acid was >99% in all cases. No detectable levels of other organic acids (formic, succinic acid) and ethanol were found.

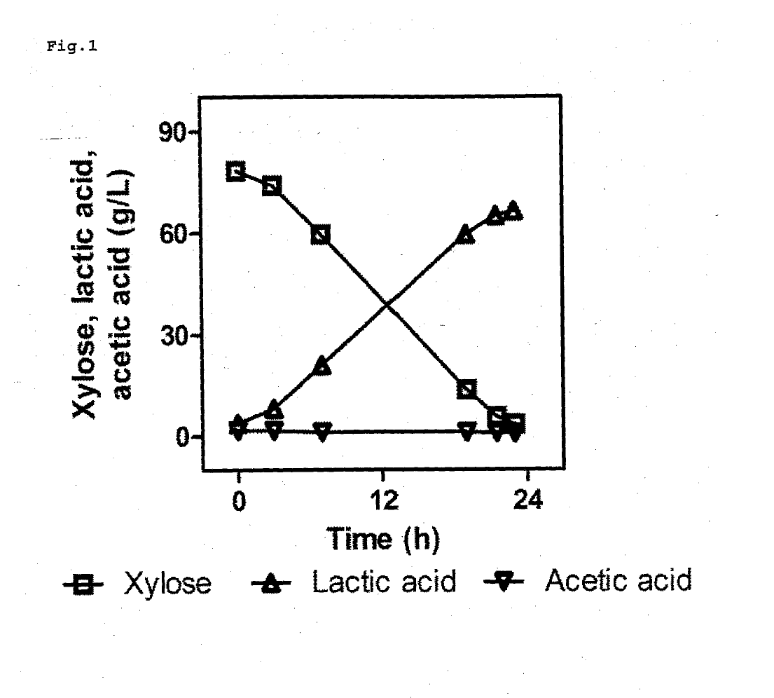

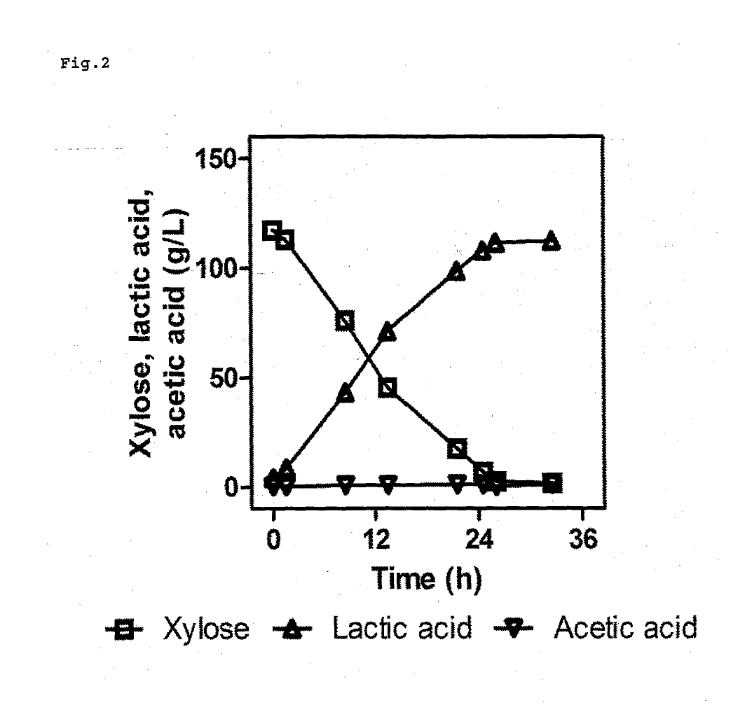

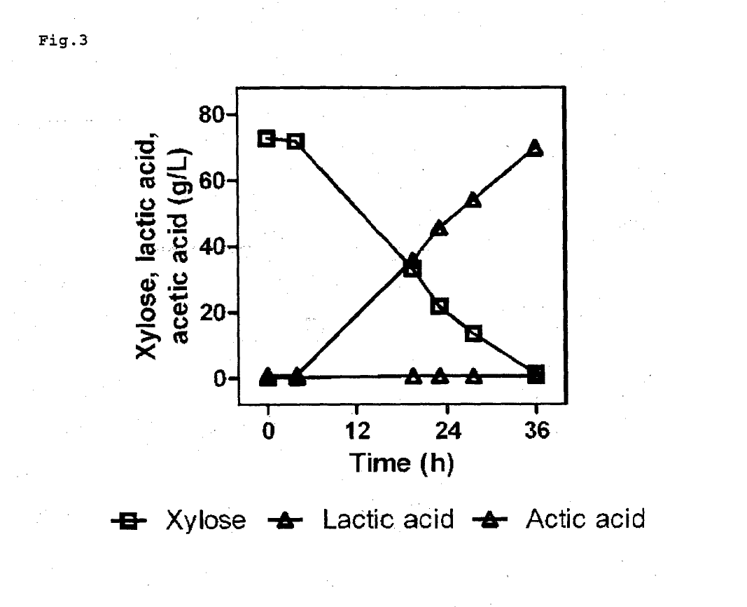

In batch fermentations, 66 g/L lactic acid was produced from xylose by B. coagulans JI12 with a yield of 91% and a productivity of 2.9 g/L/h (FIG. 1) and 101 g/L lactic acid was produced by B. coagulans C106 within 26 h giving a yield of 94% and a productivity of 3.7 g/L/h (FIG. 2). B. coagulans WCP10-4 produced 70 g/L of lactic acid from 75 g/L of xylose within 36 h with a yield of 96% and productivity of 1.9 g/L/h (FIG. 3).

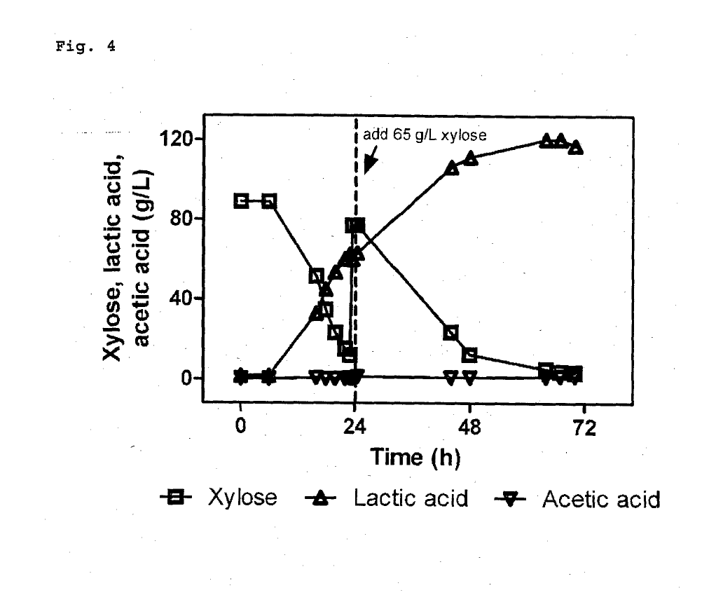

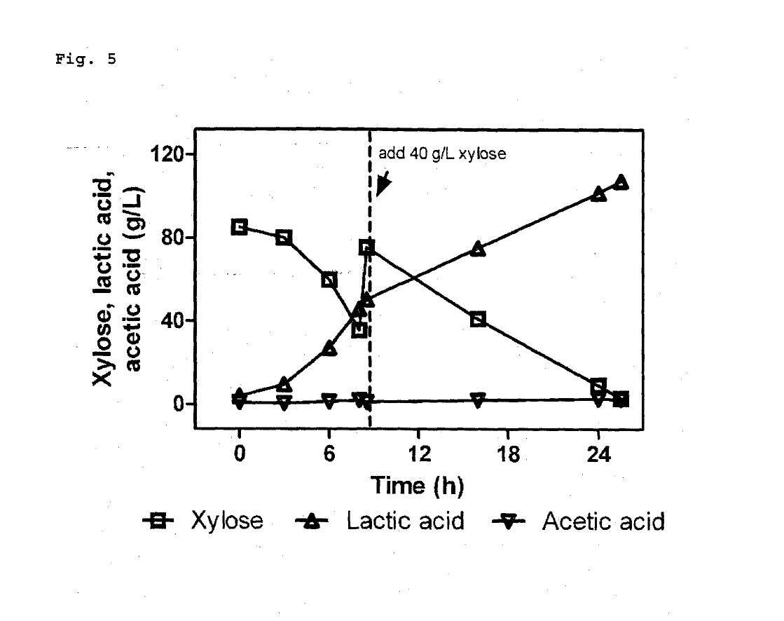

In fed-batch fermentations, B. coagulans ⁻JI12 gave a lactic acid titer of 120 g/L, a yield of 91% and a productivity of 1.9 g/L/h (FIG. 4). B. coagulans C106 produced 107 g/L lactic acid within 26 h with a yield of 96% and a productivity of 4.2 g/L/h (FIG. 5).

The three isolates gave the highest lactic acid productivities among the lactic acid bacteria ever reported (Table 1). Isolates according to the invention gave higher lactic acid titers than most of the reported strains except B. coagulans 36D1. However, the higher lactic acid titer (163 g/L) of B. coagulans 36D1 was achieved in a LB medium with 7.5% (w/v) of CaCO₃, which is expensive due to the use of peptone and unfavorable for downstream processing due to the formation of large amount of calcium salts. In addition, the productivity of this strain is much lower than those of our isolates due to the longer fermentation time.

For the isolates according to the invention B. coagulans JI12, C106 and WCP10-4, only 1-2% of yeast extract was used as the nitrogen source giving high lactic acid titer and productivity. The conventional lactic acid bacteria gave much lower productivities and titers even in the nitrogen-rich media such as MRS, let alone their poor growth or inability of growing in such nitrogen-insufficient medium as the one that were used for isolates according to the invention.

It is worth mentioning that the isolate C106 according to the invention showed much higher lactic acid titer and productivity than the B. coagulans strains ever reported in the mineral salts medium, indicating that the isolate according to the invention is providing an improved means for lactic acid production.

TABLE 1
Comparison of B. coagulans C106, JI12 and WCP10-4
with other native lactic acid bacteria for lactic acid
production from xylose

| | | | | | | | | |
|---|---|---|---|---|---|---|---|---|
| | | | | Yield | | | | |
| | | [Lactic | | g/g | Lactic | | | |
| | [xylose] | acid] | Productivity | consumed | acid | Temp. | | Nitrogen |
| Organism | (g/L) | (g/L) | (g/L h) | sugar | isomer | (° C.) | pH | Sources |

| | | | | | | | | |
|---|---|---|---|---|---|---|---|---|
| B. coagulans | 117 | 100.9 | 3.72 | 93.7% | L | 50 | 6.0 | 10 g/L |
| C106 | | | | | | | | yeast |
| | | | | | | | | extract |
| | 85 + 40 | 107.4 | 4.21 | 96.4% | L | 50 | 6.0 | 20 g/L |
| | | | | | | | | yeast |
| | | | | | | | | extract |
| B. coagulans | 78.4 | 66.4 | 2.89 | 91.1% | L | 50 | 6.0 | 10 g/L |
| JI12 | | | | | | | | yeast |
| | | | | | | | | extract |
| | 89 + 65 | 120.3 | 1.87 | 91.4% | L | 50 | 6.0 | 20 g/L |
| | | | | | | | | yeast |
| | | | | | | | | extract |
| B. coagulans | 75 | 69.8 | 1.94 | 96% | L | 50 | 6.5 | 10 g/L |
| WCP10-4 | | | | | | | | yeast |
| | | | | | | | | extract |
| B. coagulans | 120 | 89.1 | 1.86 | 88.2% | L | 50 | 6.0 | 10 g/L |
| 36D1 | | | | | | | | yeast |
| | | | | | | | | extract |
| | 120 | 102.3 | 0.71 | 86.4% | L | 50 | 6.0 | 10 g/L |
| | | | | | | | | peptone, |
| | | | | | | | | 5 g/L |
| | | | | | | | | yeast |
| | | | | | | | | extract |
| | 100 + 50 + | 163.0 | 0.75 | 87.3% | L | 50 | 6.0 | 10 g/L |
| | 50 | | | | | | | peptone, |
| | | | | | | | | 5 g/L |
| | | | | | | | | yeast |
| | | | | | | | | extract |
| B. coagulans | 30 | 20.5 | 0.43 | 80-93% | L | 50 | 5.0 | 5 g/L |
| 36D1 | | | | | | | | corn |
| | | | | | | | | steep |
| | | | | | | | | liquor |
| B. coagulans | 30 | 22.7 | 0.24 | | L | 50 | 5.0 | 5 g/L |
| 17C5 | | | | | | | | corn |
| | | | | | | | | steep |
| | | | | | | | | liquor |
| B. coagulans | 30 | 20.5 | 0.21 | | L | 50 | 5.0 | 5 g/L |
| P4-102B | | | | | | | | corn |
| | | | | | | | | steep |
| | | | | | | | | liquor |
| B. coagulans | 50 | 35 | 0.35 | n.d. | L | 54 | 6.4 | 10 g/L |
| DSM 2314 | | | | | | | | yeast |
| | | | | | | | | extract |
| L. casei | 80 | 65 | 0.27 | 80% | L | 45 | 5.7 | 5 g/L |
| subsp. | | | | | | | | yeast |
| rhamnous | | | | | | | | extract |
| (ATCC10863) | 100 | 78 | 0.20 | 78% | L | 45 | 5.7 | 5 g/L |
| | | | | | | | | yeast |
| | | | | | | | | extract |
| L. | 27 | 25.1 | 0.13 | 93% | n.d. | 45 | 5.0 | 30 g/L |
| delbrueckii | | | | | | | | yeast |
| | | | | | | | | extract |
| | 25 | 22.5 | 0.28 | 90% | n.d. | 45 | 5.0 | 27.5 g/L |
| | | | | | | | | tryptone |
| Enterococcus | 103.7 | 94.5 | 1.31 | 85% | L | 43 | 7.0 | 4.0 g |
| mundtii QU | | | | | | | | yeast |
| 25 | | | | | | | | extract, |
| | | | | | | | | 8.0 g |
| | | | | | | | | meat |
| | | | | | | | | extract, |
| | | | | | | | | 10.0 g |
| | | | | | | | | peptone |
| L. lactis | 51.2 | 24 | 0.6 | 47% | L | 37 | 6.0 | 5.0 g |
| IO-1 | | | | | | | | yeast |
| | | | | | | | | extract, |
| | | | | | | | | 5.0 g |
| | | | | | | | | Polypeptone |
| L. lactis | 70.3 | 33.3 | 0.67 | 66.7% | L | 37 | 6.0 | 5.0 g |
| IO-1 | | | | | | | | yeast |
| | | | | | | | | extract, |
| | | | | | | | | 10.0 g |
| | | | | | | | | Polypeptone |
| | 69.9 | 20.6 | n.d. | 54.7% | L | 37 | 6.0 | 5.0 g |
| | | | | | | | | yeast |
| | | | | | | | | extract, |
| | | | | | | | | 10.0 g |
| | | | | | | | | Polypeptone |
| L. pentosus | 23 | 12.9 | 0.27 | 56% | n.d. | 33 | 6.5 | 27.5 g/L |
| | | | | | | | | tryptone |
| L. xylosus | 31 | 13 | 0.24 | 48% | L | 30 | 6.4-6.5 | 7 g/l |
| | | | | | | | | yeast |
| | | | | | | | | extract, |
| | | | | | | | | 3 g/l |
| | | | | | | | | peptone |

n.d. = not determined

### LITERATURE

Patel, M. A., Ou, M. S., Harbrucker, R., Aldrich, H. C., et al., Isolation and characterization of acid-tolerant, thermophilic bacteria for effective fermentation of biomass-derived sugars to lactic acid. Applied and Environmental Microbiology 2006, 72, 3228-3235;WO2004063382; Ou, M. S., Ingram, L. O., Shanmugam, K. T., L: (+)-Lactic acid production from non-food carbohydrates by thermotolerant Bacillus coagulans. J Ind Microbiol Biotechnol 2011, 38, 599-605; Iyer, P. V., Thomas, S., Lee, Y., High-yield fermentation of pentoses into lactic acid. Applied Biochemistry and Biotechnology 2000, 84, 665-677; Thomas, S., Production of lactic acid from pulp mill solid waste and xylose using Lactobacillus delbrueckii (NRRL B445). Applied Biochemistry and Biotechnology 2000, 84, 455-468; Abdel-Rahman, M. A., Tashiro, Y., Zendo, T., Hanada, K., et al., Efficient Homofermentative L-(+)-Lactic Acid Production from Xylose by a Novel Lactic Acid Bacterium, Enterococcus mundtii QU 25. Applied and Environmental Microbiology, 77, 1892-1895; Ishizaki, A., Ueda, T., Tanaka, K., Stanbury, P. F., L-lactate production from xylose employing Lactococcus lactis IO-1. Biotechnology Letters 1992, 14, 599-604; Tanaka, K., Komiyama, A., Sonomoto, K., Ishizaki, A., et al., Two different pathways for D-xylose metabolism and the effect of xylose concentration on the yield coefficient of L-lactate in mixed-acid fermentation by the lactic acid bacterium Lactococcus lactis IO-1. Applied Microbiology and Biotechnology 2002, 60, 160-167; Tyree, R., Clausen, E., Gaddy, J., The fermentative characteristics of Lactobacillus xylosus on glucose and xylose. Biotechnology Letters 1990, 12, 51-56.

Lactic Acid Production from Glucose by B. coagulans J112, C106 and WCP10-4

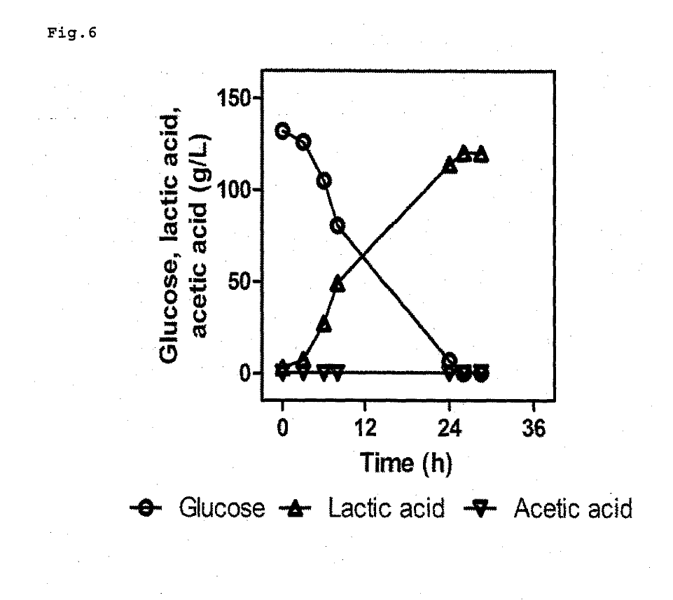

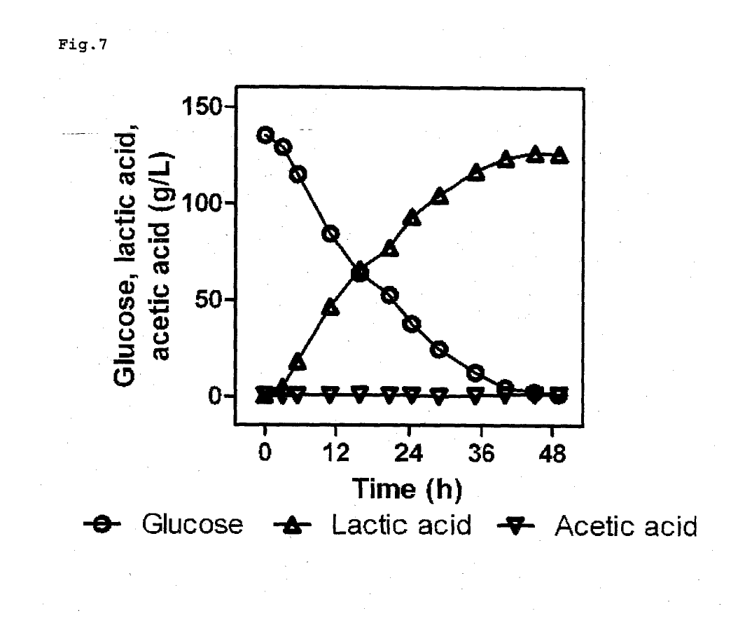

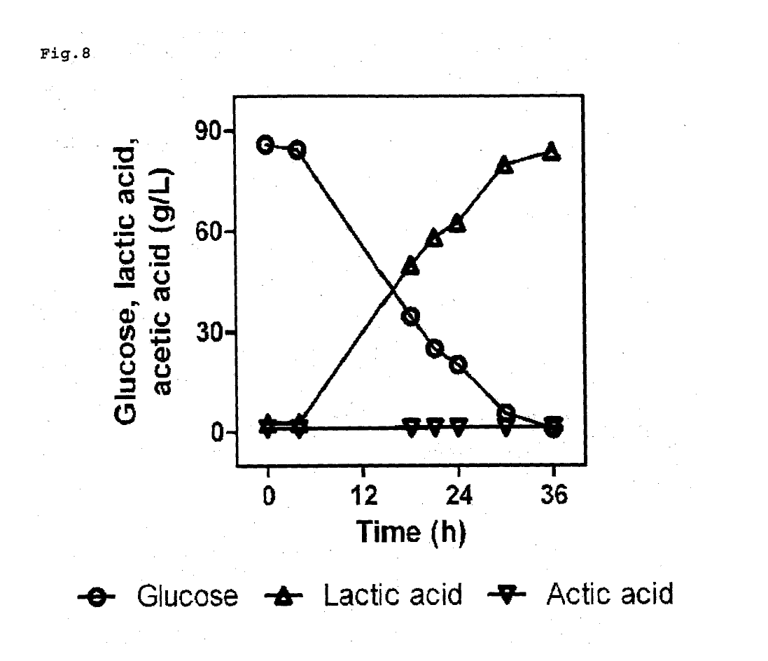

B. coagulans JI12 was grown on 130 g/L glucose in mineral salts medium supplemented with 2% yeast extract. All the glucose was consumed within 26 h giving 120 g/L lactic acid with a productivity of 4.6 _{g}/L/h and a yield of 98% (FIG. 6). B. coagulans C106 completely converted 135 g/L of glucose to lactic acid within 45 h, giving 127 g/L of lactic acid with a productivity of 2.8 g/L/h and a yield of 99% (FIG. 7). B. coagulans WCP10-4 converted 88 g/L of glucose to lactic acid within 36 h with a productivity of 2.3 g/L/h and a yield of 97% (FIG. 8). All the three isolates produced optically pure L-lactic acid (e.e. >99%) with no detectable levels of ethanol, acetic acid, formic and succinic acid.

Table 2 lists the comparison of our three isolates with other strains reported for lactic acid production from glucose. B. coagulans JI12 showed a similar productivity with B. coagulans SIM-7 but an obviously higher lactic acid titer (120 g/L) than the latter (95.5 g/L). Both lactic acid titer and productivity of B. coagulans 2-6 were similar to those of B. coagulans JI12, but this strain was cultivated in a richer medium than the one that we used for cultivating B. coagulans JI12. Therefore, for glucose fermentation to lactic acid, our isolate B. coagulans JI12 is the best in terms of high lactic acid titer, high productivity and low medium cost.

TABLE 2
Comparison of our isolates with reported strains
for lactic acid production from glucose

| | [Initial | [Lactic | | Yield g/g | Fermentation | | |
|---|---|---|---|---|---|---|---|
| | glucose] | acid] | Productivity | consumed | duration | Temp. | Nitrogen |
| Organism | (g/L) | (g/L) | (g/L h) | sugar | (h) | (° C.) | Sources |
| B. coagulans | 130 | 120.4 | 4.6 | 98% | 26 | 50 | 20 g/L |
| JI12 | | | | | | | yeast |
| | | | | | | | extract |
| B. coagulans | 135 | 126.5 | 2.8 | 99% | 45 | 50 | 20 g/L |
| C106 | | | | | | | yeast |
| | | | | | | | extract |
| B. coagulans | 88 | 83.5 | 2.3 | 97% | 36 | 50 | 10 g/L |
| VCP10-4 | | | | | | | yeast |
| | | | | | | | extract |
| B. coagulans | 126 | 91.5 | 4.0 | 97% | 23 | 56 | 185.7 g/L |
| SIM-7 | | | | | | | yeast |
| | | | | | | | autolysate |
| | 126 | 95.5 | 4.7 | 102% | 20 | 56 | 185.7 g/L |
| | | | | | | | yeast |
| | | | | | | | autolysate |
| | 144 | 101.7 | 3.1 | 95% | 33 | 56 | 185.7 g/L |
| | | | | | | | yeast |
| | | | | | | | autolysate |
| B. coagulans | 121.3 | 118 | 4.4 | 97% | 30 | 50 | 5 g/L |
| 2-6 | | | | | | | peptone, |
| | | | | | | | 10 g/L |
| | | | | | | | yeast |
| | | | | | | | extract |
| L. delbrueckii | 72 | 52 | 2.2 | 86% | 24 | 45 | 15 g/L |
| DSM 20073 | | | | | | | yeast |
| | | | | | | | extract, |
| | | | | | | | 5 g/L |
| | | | | | | | brain |
| | | | | | | | heart |
| | | | | | | | infusion, |
| | | | | | | | 5 g/L |
| | | | | | | | tryptone |

### LITERATURE

Qin, J., Zhao, B., Wang, X., Wang, L., Yu, B., Ma, Y., Ma, C., Tang, H., Sun, J., Xu, P., Non-sterilized fermentative production of polymer-grade L-lactic acid by a newly isolated thermophilic strain Bacillus sp. 2-6. PLoS One 2009, 4, e4359; Michelson, T., Kask, K., Jogi, E., Talpsep, E., et al., L (+)-Lactic acid producer Bacillus coagulans SIM-7 DSM 14043 and its comparison with Lactobacillus delbrueckii ssp. lactis DSM 20073. Enzyme and Microbial Technology 2006, 39, 861-867.

Production of Lactic Acid from Hemicellulose Hydrolysate by B. coagulans JI12

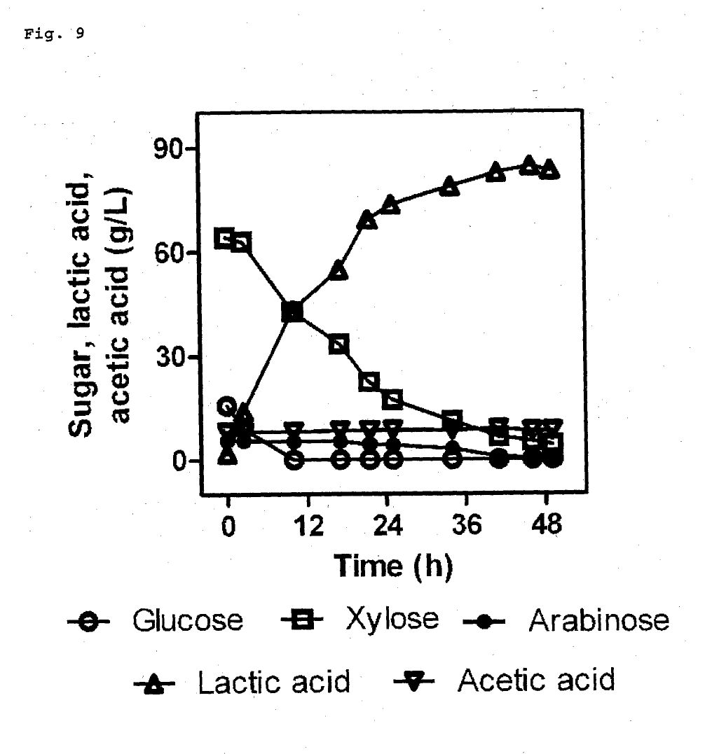

EFB hydrolysate (85.3 g sugars/L) was overlimed, neutralized and supplemented with 1% of yeast extract and 0.2% (NH₄)₂SO₄ for the fermentation without sterilization. The concentration of acetic acid from the hydrolysate was 8.1 g/L prior to fermentation. B. coagulans JI12 completely consumed all the sugars within 49 h with glucose being preferentially converted than other sugars, giving a lactic acid titer of 84.5 g/L, a yield of 96.7% and a productivity of 1.84 g/L/h (FIG. 9). It seems that the presence of acetic acid in the hydrolysate did not obviously affect the lactic acid production.

The comparison of B. coagulans JI12 with other B. coagulans strains ever reported for lactic acid production from hemicellulose hydrolysate is listed in Table 3. B. coagulans JI12 according to the invention showed improved lactic acid productivity and titer and some improvements in yield performance comparing to other strains. In addition, B. coagulans JI12 was cultivated in a medium with lower cost of nitrogen sources while other strains were cultivated in richer nitrogen media except strain 17C5, which used a diluted hydrolysate for the fermentation. It is worth mentioning that B. coagulans JI12 is able to convert all the hemicellulose sugars to L-lactic acid homofermentatively. Therefore, for lactic acid production from hemicellulose hydrolysate, the isolate B. coagulans JI12 according to the invention is the most preferred in performance relating to high lactic acid titer, high productivity and low medium cost.

TABLE 3
Comparison of B. coagulans JI12 with other B. coagulans
strains for lactic acid production from hemicellulose hydrolysates

| | | | | | Yield | | | |
|---|---|---|---|---|---|---|---|---|
| | | [Fermentab. | [Lactic | | g/g | | | |
| | | sugars] | acid] | Productivity | consumed | Temp | | Medium |
| Organism | Hydrolysate | (g/L) | (g/L) | (g/L h) | sugar | (° C.) | pH | composition |
| B. | Oil palm | 85.3 | 84.5 | 1.84 | 97% | 50 | 6.0 | Hydrolysate + |
| coagulans | empty fruit | | | | | | | 1% yeast |
| JI12 | bunch acid | | | | | | | extract + |
| | hydrolysate | | | | | | | 0.2% |
| | | | | | | | | (NH4)2SO4 |
| B. | Sugar cane | 60 (20.4 | 55.5 | 0.39 | 90% | 50 | 5.0 | 25% |
| coagulans | bagasse | from | | | | | | hydrolysate + |
| 17C5 | hemicellulose | hydrolysate, | | | | | | mineral |
| | acid | 39.6 from | | | | | | salts + |
| | hydrolysate | pure sugars | | | | | | 0.5% corn |
| | | added) | | | | | | steep |
| | | | | | | | | liquor |
| B. | Hot water- | 44 | 33 | 0.57 | 75% | 50 | 6.5 | Hydrolysate + |
| coagulans | extracted | | | | | | | 1% |
| MXL-9 | larch | | | | | | | tryptone + |
| | | | | | | | | 0.5% yeast |
| | | | | | | | | extract + |
| | | | | | | | | mineral |
| | | | | | | | | salts |
| B. | Hot water | 21.4 | 20.9 | 0.72 | 94% | 50 | 6.5 | Hydrolysate + |
| coagulans | extracted | | | | | | | 1% |
| MXL-9 | hardwood | | | | | | | tryptone + |
| | | | | | | | | 0.5% yeast |
| | | | | | | | | extract + |
| | | | | | | | | mineral |
| | | | | | | | | salts |
| | Hot water- | 45.9 | 33.5 | 0.46 | 73% | 50 | 6.5 | Hydrolysate + |
| | extracted | | | | | | | 1% |
| | Siberian | | | | | | | tryptone + |
| | larch | | | | | | | 0.5% yeast |
| | | | | | | | | extract + |
| | | | | | | | | mineral |
| | | | | | | | | salts |
| | Concentrated | 15.6 | 14.5 | 0.59 | 93% | 50 | 6.5 | Hydrolysate + |
| | green | | | | | | | 1% |
| | liquor- | | | | | | | tryptone + |
| | extracted | | | | | | | 0.5% yeast |
| | hardwood | | | | | | | extract + |
| | | | | | | | | mineral |
| | | | | | | | | salts |

### LITERATURE

WO2011053576; WO2005086670; Walton, S., Bischoff, K., van Heiningen, A., van Walsum, G., Production of lactic acid from hemicellulose extracts by Bacillus coagulans MXL-9. Journal of Industrial Microbiology & Biotechnology 2010, 37, 823-830.

Production of Lactic Acid from Corn Starch by WCP10-4 in One-Pot, One-Step Open Condition

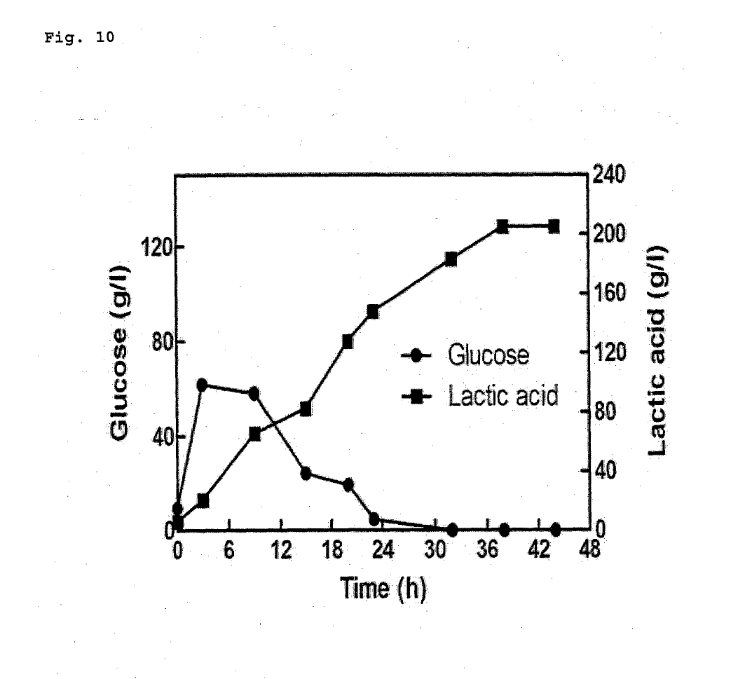

Into a 1 L unsterilized medium (20 g/L yeast extract, corn starch 200 g/L) in a 2 L fermenter were added 0.5 mL α-amylase and 5 mL glucoamylase, followed by inoculation (10%) of. B. coagulans WCP10-4. The simultaneous saccharification and fermentation. (SSF) was conducted at 50° C., 200 rpm. The pH was controlled at pH 6.0 by automatic addition of calcium hydroxide (35%, w/v). after 37 h, the lactic acid titer and productivity reached as high as 202.0 g/L and 5.4 g/L/h, respectively. No glucose was detected in the broth and the lactic acid yield was determined to be 98%; (FIG. 10).

### Summary of Results

The three isolated Bacillus coagulans strains according to the invention showed high titer, productivity and yield in producing L-lactic acid from hemicellulose sugars. The highest lactic acid productivity on xylose reached 4.2 g/L/h by Bacillus coagulans C106 and the highest lactic acid titer reached 120 g/L/h by Bacillus coagulans JI12.

For glucose fermentation, the highest lactic acid productivity reached 4.6 g/L/h by Bacillus coagulans JI12 and the highest lactic acid titer reached 127 g/L/h by Bacillus coagulans C106. Bacillus coagulans JI12 converted all the sugars in hemicellulose hydrolysate to L-lactic acid homofermentatively with a productivity of 1.84 g/L/h, which is the highest among the lactic acid productivities on hemicellulose hydrolysate ever reported. The combined use of Bacillus coagulans JI12 and Bacillus coagulans C106 is expected to synergistically improve the lactic acid production efficiency from lignocellulose hydrolysates.

### Examples of Lactic Acid Production

### Example 1

Into 1 L of mineral salts medium containing 85 g/L of xylose and 20 g/L of yeast extract was inoculated 10% of Bacillus coagulans C106. The seed culture was prepared in 100 ml mineral salts medium containing 5% of xylose, 1% of yeast extract and 3% of CaCO3 in 250-ml conical flasks at 50° C. and 200 rpm for 1 day. NaOH (15 M) was automatically added to neutralize the produced lactic acid to maintain the pH at 6.0 during the fermentation. The xylose concentration dropped to lower than 40 g/L after 8 h, then another 40 g/L of xylose was added. All the added xylose was converted to L-lactic acid within 26 h, giving a lactic acid titer of 107.4 g/L, a productivity of 4.2 g/L/h and a yield of 96.4% with an optical purity of >99%.

### Example 2

Into 1 L of mineral salts medium containing 130 g/L of glucose and 20 g/L of yeast extract was inoculated 10% of Bacillus coagulans JI12. The seed culture was prepared in 100 ml mineral salts medium containing 5% of glucose, 1% of yeast extract and 3% of CaCO₃ in 250-ml conical flasks at 50° C. and 200 rpm for 1 day. NaOH (10 M) was automatically added to neutralize the produced lactic acid to maintain the pH at 6.0 during the fermentation. After 26 h, all glucose was consumed, giving a L-lactic acid titer of 120.4 g/L, a productivity of 4.6 g/L/h and a yield of 98% with an optical purity of >99%.

### Example 3

EFB hydrolysate was over-limed to pH 11 using Ca(OH)₂ followed by centrifugation to remove the precipitant and neutralization to pH 6.0 using H₂SO₄. The treated EFB hydrolysate contained 85.3 g/L of total sugars (15.7 g/L glucose, 5.5 g/L arabinose and 64.1 g/L xylose) and 8.1 g/L of acetic acid. Into 500 ml of the treated EFB hydrolysate was supplemented with 1% of yeast extract and 0.2% of (NH₄)₂SO₄ followed by inoculation of 10% of Bacillus coagulans JI12. The seed culture was prepared in 50 ml mineral salts medium containing 5% of xylose, 1% of yeast extract and 3% of CaCO₃ in 250-ml conical flasks at 50° C. and 200 rpm for 1 day. The cells were harvested by centrifugation and re-suspended in the liquid of the same compositions with the fermentation medium for use as the inoculum. NaOH (15 M) was used to neutralize the produced lactic acid to maintain the pH at 6.0 during the fermentation. After 49 h, all the sugars were converted giving a L-lactic acid titer of 84.5 g/L with a productivity of 96.7% and a yield of 1.84 g/L/h.

### Example 4

Into a 1 L unsterilized medium (20 g/L yeast extract, corn starch 200 g/L) in a 2 L fermenter were added 0.5 mL α-amylase and 5 mL glucoamylase, followed by inoculation (10%) of B. coagulans WCP10-4. The simultaneous saccharification and fermentation (SSF) was conducted at 50° C., 200 rpm, pH 6.0. After 37 h, the lactic acid titer, productivity and yield reached 202.0 g/L, 5.4 g/L/h and 98%, respectively. All glucose was utilized upon completion of the fermentation.

### Second Set of Examples

Efficient Production of L-Lactic Acid by a Newly Isolated Thermophilic Bacillus coagulans WCP10-4 with High Glucose Tolerance

Materials and Methods

Materials and Enzymes

Corn starch (starch content 100%) was purchased from Sigma-Aldrich. Liquozyme SC DC (α-amylase, 240 KNU/g, KNU, the amount of enzyme that breaks down 5.26 g of starch per hour under Novozyme's assay conditions for α-amylase) and Spirizyme Fuel (glucoamylase, 750 AGU/g, AGU, the amount of enzyme that hydrolyzes 1 μmol of maltose per minute under specified conditions) were gifts from Novozymes, Denmark.

Isolation of Thermophilic Strains

Soil samples were collected from various locations and suspended in 50 mL of enrichment medium (10 g/L yeast extract, 20 g/L glucose) for overnight at 50° C. without shaking. The enriched samples were serially diluted and plated on agar plates (20 g/L yeast extract, 100 g/L glucose, 0.4 g/L bromocresol green and 1.5% agar) at 50° C. After 48 h of incubation, representative colonies with yellow circles were picked up and individually incubated in 100 mL of medium (20 g/L yeast extract, 100 g/L glucose, 50 g/L CaCO3) for 48 h. The supernatants were collected by centrifugation for HPLC analysis to determine lactic acid concentrations in broths. One bacterial strain (WCP10-4) that grew well in the liquid medium and produced the highest concentration of L-lactic acid was finally isolated from garden soil samples.

For identification of the bacterial isolate WCP10-4, its partial 16S rRNA gene sequence was amplified using the universal primers 27F (5′-AGAGTTTGATCCTGGCTCAG-3′) and 1492R (5′-TTACCTTGTTACGACTT 3′) and compared with those in the GenBank database through BLAST sequence analysis.

Fermentation Conditions

WCP10-4 was stored at −80° C. in the medium composed of 75% of the medium and 25% of glycerol. A seed culture was prepared by adding 250 μL of stock culture to a sterile tube containing 5 mL of the above medium. The tube was incubated at 50° C. and 200 rpm for 16 h, followed by adding the broth into 100 mL of fresh medium in a 500 mL Erlen-meyer flask and shaking at 50° C. for 24 h. The seed culture thus prepared was inoculated into 1 L fresh medium in a 2 L fermenter (Biostat® B plus, Statorius Stedium Biotech, Goettingen, Germany) for L-lactic acid production at 50° C. and 200 rpm. The medium for preparing seed cultures was sterilized by autoclaving but the medium for use in fermenter was not. Calcium hydroxide (35%, w/v) was used to control the pH during fermentation. The effect of pH was investigated by altering only pH while maintaining other conditions unchanged. To investigate the glucose tolerance of WCP10-4, batch fermentations were conducted at pH6.0 by changing only the glucose concentration (80-280 g/L) in the medium.

Simultaneous Saccharification and Fermentation (SSF) of Corn Starch

The SSF of corn starch was carried out in a 2 L fermenter containing 1 L medium (20 g/L yeast extract, 200 g/L corn starch), 0.5 mL α-amylase and 5 mL glucoamylase at 50° C. and 200 rpm. The pH was controlled at 5.5 or 6.0 using Calcium hydroxide (35%, w/v). Except the medium for preparing seed cultures, no sterilization was conducted for the media and fermenter.

Analytical Methods

Glucose, lactic acid, acetic acid and ethanol were analyzed by HPLC (Shimadzu, LC-10AT, Kyoto, Japan) equipped with a Bio-Rad. Aminex HPX-87H column (300×7.8 mm, Bio-Rad, Herculse, Calif., USA) and a refractive index detector (SPD-10A, Shimadzu) at 50° C. The mobile phase was 12 mM H2SO4 at 0.65 mL/min. Optical purity of lactic acid was checked on the same HPLC equipped with a MCI(R) GEL CRS15W column (50×4.6 mm, Mitubishi Chemical, Tokyo, Japan) at 30° C. using 2 mM CuSO4 as the mobile phase at 0.4 mL/min.

Results

Isolation and Identification of Thermophilic Lactic Acid Producer

According to the invention a strain numbered WCP10-4 was surprisingly found to show best performance in producing lactic acid. Its 16S rDNA sequence analysis indicated a 100% identity to that of Bacillus coagulans 36D1. Therefore, this strain was identified as Bacillus coagulans WCP10-4. Under anaerobic condition, Bacillus coagulans WCP10-4 produced primarily L-lactic acid at an enantiomeric excess (ee) of 99.8% with the formation of trace amounts (<1 g/L) of acetic acid and ethanol. Under aerobic conditions, the strain produced lactic acid as the primary product but with acetic acid concentration increased up to 4 g/L. These product profiles are distinguished from those of known Bacillus coagulans 36D1 whose primary product is acetic acid under aerobic conditions.

Effects of pH on L-Lactic Acid Production

The effect of pH on lactic acid production was performed with 1 L medium in a 2 L fermenter. From Table 1 it is seen that at all pH levels, L-lactic acid was produced at over 35 g/L with an optical purity of almost 100% and a yield of over 98%. The optimal pH was between 6.0-6.5, at which 120 g/L of glucose was completely consumed within 22 h, giving an average L-lactic acid productivity of 5.3 g/L/h. At pH 7.0, the strain was still able to consume all glucose but at a lower productivity of 3.1 g/L/h. The lactic acid productivity started to decrease dramatically when pH was below 6. At pH5.0, the strain even failed to consume all the initial glucose. The cell growth was also poor at pH 5.0 than at pH6.0. It has been proposed that the metabolic energy produced by anaerobic acidogenic fermentation is not enough for maintaining a proton gradient between the cells and their external environment (Baronofsky J J, Schreurs W J, Kashket E R (1984) Uncoupling by acetic acid limits growth and acetogenesis by Clostridium thermoaceticum. Appl. Environ Microbial 48(6): 1134-9). The optimal pH range of Bacillus coagulans WCP10-4 is very similar to that of most other lactic acid bacteria.

TABLE 1
Effect of pH on L-lactic acid production by Bacillus coagulans WCP10-4^{a}.

| | | Fermentation | | | | |
|---|---|---|---|---|---|---|
| | Initial glucose | time | L-lactic acid titer | Yield^{b} | Productivity | |
| pH | (g/L) | (h) | (g/L) | (g/g) | (g/L/h) | OD₆₀₀ |
| 5.0 | 120.5 ± 1.9 | 42 | 35.5 ± 2.5 | 98.5 ± 0.2 | 0.83 ± 0.06 | 8.5 ± 0.6 |
| 5.5 | 120.2 ± 2.5 | 36 | 108.8 ± 1.2 | 98.5 ± 0.5 | 3.02 ± 0.05 | 14.2 ± 0.4 |
| 6.0 | 120.3 ± 2.2 | 22 | 115.7 ± 3.2 | 99.3 ± 0.5 | 5.28 ± 0.05 | 16.5 ± 0.5 |
| 6.5 | 120.5 ± 1.5 | 22 | 115.5 ± 1.2 | 98.4 ± 0.5 | 5.28 ± 0.05 | 16.0 ± 0.4 |
| 7.0 | 120.4 ± 3.0 | 36 | 110.2 ± 1.5 | 98.1 ± 0.5 | 3.1 ± 0.1 | 15.5 ± 0.3 |

^{a}The experiment was performed in a 2 L fermenter containing 1 L medium (glucose 240 g/L, yeast extract 20 g/L) at 50° C., 200 rpm.
^{b}Yield = g L-lactic acid/g glucose consumed.

Effects of Glucose Concentration on L-Lactic Acid Production

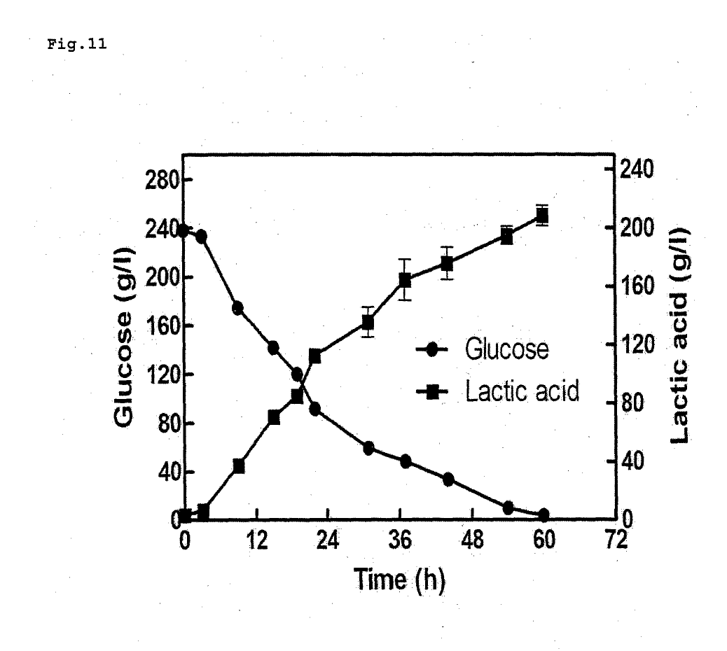

The effect of initial glucose concentration on lactic acid production was performed with 1 L medium-in a 2 L fermenter (Table 2). It is seen that even when the glucose concentration was up to 240 g/L, no significant changes of cell growth and L-lactic acid production rates were observed compared to those at lower glucose concentrations. All glucose was consumed by Bacillus coagulans WCP10-4 when its concentration was not more than 240 g/L. The highest L-lactic acid titer (210 g/L) was obtained at 240 g/L of glucose with a productivity of 3.5 g/L/h (FIG. 11). The highest lactic acid productivity (5.28 g/L/h) was observed at 80-120 g/L of glucose. At glucose concentrations below 240 g/L, the lactic acid yield was over 95%. When glucose concentration was increased to 280 g/L, lactic acid production rate was obviously and only part of the glucose was consumed, indicating a significant substrate inhibition.

TABLE 2
Effect of glucose concentration on L-lactic
acid production by Bacillus coagulans WCP10-4^{a}.

| | | L-lactic | | | |
|---|---|---|---|---|---|
| Initial | | acid | | | |
| glucose | Fermentation | titer | Yield^{b} | Productivity | |
| (g/L) | time (h) | (g/L) | (g/g) | (g/L/h) | OD₆₀₀ |
| 80.4 ± 3.5 | 15 | 78.6 ± 2.5 | 98.2 ± 0.1 | 5.24 ± 0.1 | 12.5 ± 0.2 |
| 120.6 ± 2.8 | 22 | 115.7 ± 3.2 | 98.5 ± 0.1 | 5.28 ± 0.05 | 16.5 ± 0.4 |
| 160.4 ± 3.4 | 36 | 145.4 ± 3.8 | 97.8 ± 0.3 | 4.14 ± 0.1 | 21 ± 0.1 |
| 200.6 ± 1.5 | 45 | 178.6 ± 5.5 | 96.4 ± 0.4 | 3.95 ± 0.2 | 22 ± 0.3 |
| 240.4 ± 1.9 | 60 | 210.5 ± 6.8 | 95.5 ± 0.4 | 3.5 ± 0.1 | 22 ± 0.5 |

^{a}The experiment was conducted in a 2 L fermenter containing 1 L medium (yeast extract 20 g/L) at pH 6.0, 50° C., 200 rpm.
^{b}Yield = g L-lactic acid/g glucose consumed.

For commercial production of lactic acid, high titer, yield and productivity are essential to reduce the downstream separation costs. Efficient fermentation at high initial substrate concentrations is very helpful for getting high lactic acid titer to improve the process economy. However, only a few lactic acid bacteria such as Lactobacillus paracasei subsp. paracasei CHB2121 (Moon S K, Wee Y J, Choi G W (2012) A novel lactic acid bacterium for the production of high purity l-lactic acid, Lactobacillus paracasei subsp. paracasei CHB2121. J Biosci Bioeng 114(2): 155-9) and Lactobacillus casei mutant G-03(Ge X Y, Yuan J, Qin H, Zhang W G (2011) Improvement of L-lactic acid production by osmotic-tolerant mutant of Lactobacillus casei at high temperature. Appl Microbiol Biotechnol 89(1): 73-8) have been reported to be able to tolerate high glucose concentrations. The reported thermophilic lactic acid producers are hardly able to tolerate 130 g/L of glucose or xylose (Table 3). Therefore, fed-batch fermentation has to be adapted to get high lactic acid titer (>150 g/L) when using these strains. For example, Bacillus coagulans 36D1 produced 182.2 g/L of L-lactic acid (Ou M S, Ingram L O, Shanmugam K T (2011) L: (+)-Lactic acid production from non-food carbohydrates by thermotolerant Bacillus coagulans. J Ind Microbiol Biotechnol 38(5): 599-605) and Bacillus sp. 2-6 produced 172.5 g/L of L-lactic acid (Qin J, Zhao B, Wang X, Wang L, Yu B, Ma Y, Ma C, Tang H, Sun J, Xu P (2009) Non-sterilized fermentative production of polymer-grade L-lactic acid by a newly isolated thermophilic strain Bacillus sp. 2-6. both were lower than 210 g/L obtained using Bacillus coagulans WCP10-4 in single batch. So Bacillus coagulans WCP10-4 is commercially more attractive in terms of its higher substrate tolerance thus higher lactic acid titer.

TABLE 3
Comparison of L-lactic acid titers of different strains on glucose

| | | | | | | Fermentation | pH and |
|---|---|---|---|---|---|---|---|
| | Fermentation | Initial | Titer | Productivity | Yield | temperature | buffering |
| Organism | mode | glucose (g/L) | (g/L) | (g/L/h) | (%) | (° C.) | agent |
| Bacillus | Fed-batch | 100 | 182.2 | 0.84 | 92.3 | 50 | 6.0, KOH |
| coagulans | | | | | | | |
| 36D1 | | | | | | | |
| Lactobacillus | Fed-batch | 50 | 210 | 2.2 | 97 | 37 | 6.2, |
| lactis BME5- | | | | | | | CaCO₃ |
| 18M | | | | | | | |
| Bacillus | Fed-batch | 130 | 172.5 | 2.88 | 95.8 | 50-55 | 5.6, |
| coagulans 2-6 | | | | | | | CaCO₃ |
| Lactobacillus | Batch | 210 | 198.2 | 5.5 | 94.4 | 41 | CaCO₃ |
| casei G-03 | | | | | | | |
| Lactobacillus | Fed-batch | 90 | 180 | 2.14 | 90.3 | 42 | 6.25, |
| casei LA-04-1 | | | | | | | NH₄OH |
| Lactobacillus | Fed-batch | 125 | 170 | 2.7 | | 42 | 6.25, |
| casei LA-04-1 | | | | | | | CaOH and |
| | | | | | | | NH₄OH |
| Lactobacillus | Batch | 200 | 192 | 3.99 | 95 | 38 | 6.5, |
| paracasei | | | | | | | NaOH |
| subsp. paracasei | | | | | | | |
| iCHB2121 | | | | | | | |
| Bacillus sp. | Fed-batch | 80 | 225 | 1.04 | 99.3 | 45 | 9.0, |
| WL-S20 | | | | | | | NaOH |
| Bacillus | Batch | 240 | 210 | 3.5 | 95.5 | 50 | 6.0, CaOH |
| coagulans | | | | | | | |
| WCP10-4 | | | | | | | |

With regard to the strains not isolated according to invention reference is made to the listed references for the following strains:

36D1: Ou M S, Ingram L O, Shanmugam K T (2011) L: (+)-Lactic acid production from non-food carbohydrates by thermotolerant Bacillus coagulans. J Ind Microbiol Biotechnol 38(5): 599-605

BME5-18M: Bai D M, Wei Q, Yan Z H, Zhao X M, Li X G, Xu S M (2003) Fed-batch fermentation of Lactobacillus lactis for hyper-production of L-lactic acid. Biotechnol Lett 25(21): 1833-1835

2-6: Qin J, Zhao B, Wang X, Wang. L, Yu B, Ma Y, Ma C, Tang H, Sun J, Xu P (2009) Non-sterilized fermentative production of polymer-grade L-lactic acid by a newly isolated thermophilic strain Bacillus sp. 2-6. PLoS One 4(2): e4359

LA-04-1 (1^{st} shown): Li Z, Ding S, Tan T (2006) L-lactic acid production by Lactobacillus casei fermentation with corn steep liquor-supplemented acid-hydrolysate of soybean meal. Biotechnol J 1(12): 1453-1458

LA-04-1 (2^{nd} shown): Li Z, Lu J, Zhao L, Xiao K, Tan T (2010) Improvement of L: -Lactic Acid Production under Glucose Feedback Controlled Culture by Lactobacillus rhamnosus. Appl Biochem Biotechnol 162(6): 1762-1767

CHB2121: Moon S K, Wee Y J, Choi G W (2012a) A novel lactic acid bacterium for the production of high purity l-lactic acid, Lactobacillus paracasei subsp. paracasei CHB2121. J Biosci Bioeng 114(2): 155-9

WL-S20: Meng Y, Xue Y, Yu B, Gao C, Ma Y (2012) Efficient production of l-lactic acid with high optical purity by alkaliphilic Bacillus sp. WL-S20. Bioresour Technol 116334-339

Simultaneous Saccharification and Fermentation of Corn Starch

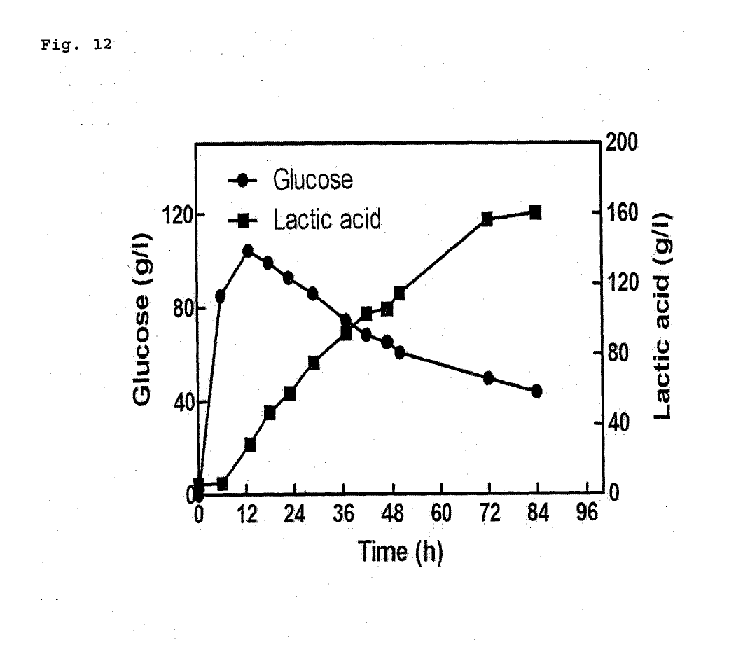

As the optimal pH for amylase and glucoamylase is 4.5-5.5 and Bacillus coagulans WCP10-4 showed good productivity (3.0 g/L/h) at pH5.5, the SSF was first performed at pH5.5. As shown in FIG. 12, glucose was quickly accumulated in the beginning due to the low consumption by the lactic acid bacterium, which might need some time to adapt the new environment. Lactic acid was rapidly produced (3.3 g/L/h) at 6 h and the glucose concentration reached the highest at 13 h. The final lactic acid titer was 160.5 g/L with a productivity of 1.9 g/L/h, leaving 43.7 g/L of glucose unconsumed in the end, which might be caused by the lower pH environment.

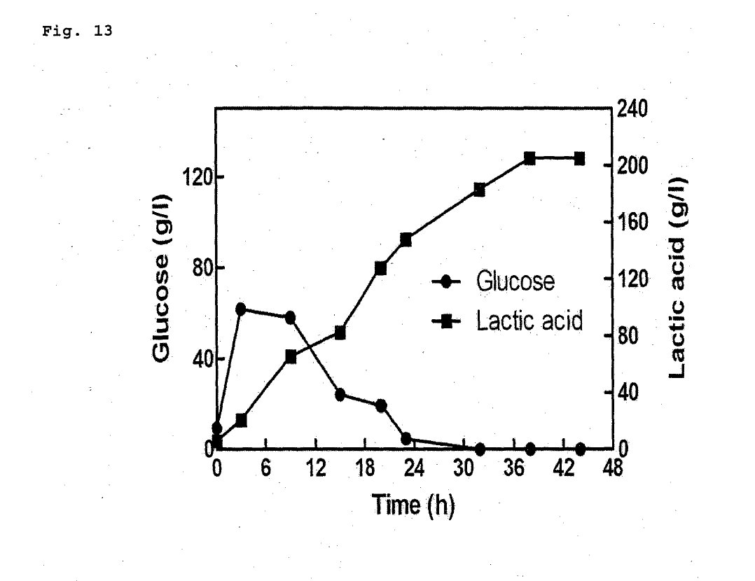

To favor the lactic acid fermentation, the SSF was performed at pH 6.0 which is more near to the optimal pH of the strain (FIG. 13). The lactic acid production was markedly improved compared to the case at pH5.5. The final lactic acid titer and productivity were as high as 202.0 g/L and 5.4 g/L/h, respectively (FIG. 13). No glucose was detected in the broth and the lactic acid yield was determined to be 98%.

Although lignocellulose has attracted much attention as a renewable and abundant resource for producing chemicals such as lactic acid, its cost-effective pretreatment to get fermentable sugars at an acceptable price is still a big challenge. Therefore, in the foreseeable future, starchy materials will remain to be the major carbon sources for producing lactic acid (John R P, G S A, Nampoothiri K M, Pandey A (2009) Direct lactic acid fermentation: focus on simultaneous saccharification and lactic acid production. Biotechnol Adv 27(2): 145-52). The conventional lactic acid production from starchy materials needs several steps including gelatinisation and liquefaction at 90-130° C., enzymatic saccharification at 50-70° C. and fermentation at 30-40° C. (Reddy G, Altaf M, Naveena B J, Venkateshwar M, Kumar E V (2008) Amylolytic bacterial lactic acid fermentation—a review. Biotechnol Adv 26(1): 22-34). Recently, direct starch fermentation using amylolytic strains or SSF has received much attention in lactic acid production, but the lactic acid titer of amylolytic bacteria is usually low due to the low expression level of amylase (John R P, G S A, Nampoothiri K M, Pandey A (2009) Direct lactic acid fermentation: focus on simultaneous saccharification and lactic acid production. Biotechnol Adv 27(2): 145-52; Reddy G, Altaf M, Naveena B J, Venkateshwar M, Kumar E V (2008) Amylolytic bacterial lactic acid fermentation—a review. Biotechnol Adv 26(1): 22-34). For SSF, high lactic acid titer is usually achievable but sterilization is essential for traditional mesophilic lactic acid bacteria (Wang L, Zhao B, Liu B, Yang C, Yu B, Li Q, Ma C, Xu P, Ma Y (2010) Efficient production of L-lactic acid from cassava powder by Lactobacillus rhamnosus. Bioresour Technol). Here we demonstrated that Bacillus coagulans WCP10-4 converted corn starch to optically pure L-lactic acid with high titer, yield and productivity in one-step without requirement of sterilizing the medium and fermenter prior to fermentation (open fermentation).

The Bacillus coagulans strains according to the invention were deposited on Sep. 26, 2012 at the American Type Culture Collection (ATTC®), (ATCC® Patent Depository, 10801 University Blvd., Manassas, Va. 20110, USA) as Bacillus coagulans JI12 with ATTC®Patent Deposit Designation PTA-13253, as Bacillus coagulans C106 with ATTC®Patent Deposit Designation PTA-13254 and as Bacillus coagulans WCP 10-4 with ATTC®Patent Deposit Designation PTA-13255.

1. Bacillus coagulans strain C106 deposited under Accession No. PTA-13254 or a functional mutant thereof.

2. Bacillus coagulans strain JI 12 deposited under Accession No. PTA-13253 or a functional mutant thereof.

3. Bacillus coagulans strain WCP10-4 deposited under Accession No. PTA-13255 or a functional mutant thereof.

4. A Bacillus coagulans strain according to claim 1, which is biologically pure.

5. A method of producing lactic acid from a carbohydrate source, comprising the steps of:
(i) contacting said carbohydrate source with at least one Bacillus coagulans strain selected from the group consisting of Bacillus coagulans strain C106 deposited under Accession No. PTA-13254; Bacillus coagulans strain JI 12 deposited under Accession No. PTA-13253; Bacillus coagulans strain WCP10-4 deposited under Accession No. PTA-13255; and a functional mutant thereof to form lactic acid;
(ii) isolating said lactic acid.

6. The method of claim 5, further comprising, prior to step (ii), the step of incubating the carbohydrate source with at least one Bacillus coagulans strain selected from the group consisting of Bacillus coagulans strain C106 deposited under Accession No. PTA-13254; Bacillus coagulans strain JI 12 deposited under Accession No. PTA-13253; Bacillus coagulans strain WCP10-4 deposited under Accession No. PTA-13255; and a functional mutant thereof.

7. The method of claim 5, wherein said carbohydrate source is selected from the group consisting of lignocellulose, hemicellulose, D/L-glucose, D/L-galactose, D/L-mannose, D/L-arabinose, D/L-lyxose, D/L-ribose, D/L-xylose, D/L-ribulose, D/L-xylulose, saccharides and polysaccharides thereof.

8. The method of claim 7 wherein the carbohydrate source is L-xylose.

9. The method of claim 5, wherein the said at least one Bacillus coagulans strain is C106 deposited under Accession No. PTA-13254.

10. The method of claim 7, wherein two Bacillus coagulans strains are incubated with said carbohydrate source.

11. The method of claim 10, wherein said two Bacillus coagulans strains are C106 deposited under Accession No. PTA-13254 and JI 12 deposited under Accession No. PTA-13253.

12. The method of claim 5, wherein the lactic acid is L-lactic acid.

13. The method of claim 12, wherein the optical purity of said isolated L-lactic acid is greater than 99%.

14. The method of claim 12, wherein the titer of isolated L-lactic acid is in the range selected from the group consisting of 50 to 150 g/L, 60 to 140 g/L, 70 to 130 g/L, 80 to 130 g/L, 90 to 130 g/L, and 100 to 130 g/L.

15. The method of claim 12, wherein the production of L-lactic is in the range selected from the group consisting of 1 to 8 g/L/hour, 2 to 7 g/L/hour, 3 to 6 g/L/hour, and 4 to 5 g/L/hour.

16. The method of claim 12, wherein the yield of said isolated L-lactic acid is greater than 90%.

17. The method of claim 6 wherein said at least one Bacillus coagulans strain is incubated with said carbohydrate source at a temperature of from 50-55° C.

18. The method of claim 6 wherein said at least one Bacillus coagulans strain is incubated with said carbohydrate source for a period of time from 24 to 72 hours.

19. A seed inoculum comprising Bacillus coagulans strain C106 deposited under Accession No. PTA-13254 or a functional mutant thereof.

20. A seed inoculum comprising Bacillus coagulans strain JI 12 deposited under Accession No. PTA-13253 or a functional mutant thereof.

21. A seed inoculum comprising Bacillus coagulans strain WCP10-4 deposited under Accession No. PTA-13255 or a functional mutant thereof.

22. The seed inoculum of claim 19, further comprising a carrier.

23. The seed inoculum of claim 22, wherein the carrier is an aqueous medium.

24. A kit comprising at least one Bacillus coagulans strain as claimed in claim 1, together with instructions for use.

25. A kit comprising at least one seed inoculum as claimed in claim 19, together with instructions for use.
